(19) United States
(12) Patent Application Publication
(10) Pub. No.: US 2003/0099934 A1
(43) Pub. Date: May 29, 2003

(54) Chemically modified hiv envelope glycoprotein

(76) Inventors: Florence Boudet, Lyon (FR); Michel Chevalier, Beaurepaire (FR); Jean Dubayle, Theize (FR); Raphaelle El Habib, Chaponost (FR)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/169,442

(22) PCT Filed: Dec. 27, 2000
(86) PCT No.: PCT/FR00/03690

(30) Foreign Application Priority Data
Jan. 4, 2000 (FR) 00/00059

(51) Int. Cl. C12Q 1/70; C07H 21/04; C12N 7/00; C12P 21/02; C12N 5/06; C07K 14/16

(52) U.S. Cl. 435/5; 435/69.3; 435/320.1; 435/325; 435/235.1; 536/23.72; 530/395

(57) ABSTRACT

The present invention relates to an envelope glycoprotein of HIV in purified form which can be obtained by a method comprising the following steps: (1) production of an envelope glycoprotein in purified form, (2) reduction of at least one disulfide bridge of the glycoprotein of step (1), (3) alkylation of at least two free sulfhydryl groups, (4) optionally, oxidation of the remaining free sulfhydryl groups, (5) denaturation and (6) renaturation, and to its use in a vaccine against HIV which can be used for inducing antibodies which neutralize HIV in a human individual, therapeutically or prophylactically.

The present invention relates to a novel antigen and to its use in a vaccine against HIV, and relates more particularly to a chemically modified envelope glycoprotein of HIV, capable of inducing antibodies which neutralize primary isolates of HIV.

These studies have been cofinanced by the ANRS.

Over the last ten years, several vaccines against HIV have been proposed and tested in monkeys or in humans. None of the vaccines proposed to date has provided a totally satisfactory solution. The major obstacles, namely the great genetic variability of the virus (Saragosti S., 1997, Virologie, 1: 313-320) and the low exposure to the immune system of viral epitopes which can be neutralized, considerably slow down the development of a vaccine which allows the induction of neutralizing immunity.

The envelope glycoprotein of HIV, which is required in order to confer on the virus its infectious nature, represents the target for neutralizing antibodies. These characteristics have made the latter a subject of intense investigations. It has been shown that the envelope glycoprotein of HIV is an oligomer composed of an extracellular domain, gp120, and of a transmembrane domain, gp41 (Gallaher et al., AIDS Research & Human Retroviruses 11(2): 191-202, 1995). Leonard et al. have shown that gp160 comprises 20 cysteine residues forming 10 disulfide bridges.

Various approaches directed toward producing antibodies which neutralize the primary isolates of HIV have been proposed, but none has provided a really satisfactory solution.

Parren et al. have demonstrated a correlation between the production of antibodies which can neutralize, in vitro, the infection of cells with HIV and the oligomeric nature of gp120 (J. of Virology, 72, 3512-3519, 1998). In addition, Earl et al. have shown that antibodies specific for the oligomeric structure of gp160 can be generated and participate, in fact, in a neutralizing effect against the in vitro infection of cells with HIV (PNAS 87, 648-652, 1990).

Several authors have proposed modifying the structure of gp160 with the aim of producing a protein which is closer to the one present at the surface of the virus during the step of HIV binding and of cell membrane fusion, and/or exposing initially hidden epitopes.

A. Benjouad et al. (J. Virology, p 2473-2483, 1992) have proposed the use of a gp160 which has been enzymatically deglycosylated in order to induce neutralizing antibodies. The results obtained show that the antibodies derived from antisera produced against a desialylated gp160 neutralize the infectious power of HIV-1 (TCLA) and inhibit the formation of a syncytium between the cells infected with HIV-1 and the noninfected CD4+ cells.

R. A. LaCasse et al. (Science, 283: 357-362, Jan. 15, 1999) have described the preparation of a vaccine comprising whole cells fixed with formaldehyde, which is thought to reproduce the transient envelope protein/CD4/coreceptor structure present during HIV infection. The use of such a preparation would, in a transgenic mouse model, cause the neutralization of many primary isolates of HIV. It has not been possible to reproduce this experiment.

The neutralizing antibody responses, as described in the prior art mentioned above, have the drawback either of being specific for a given serotype, or of being incapable of causing the neutralization of primary isolates of HIV. Because of the very great genetic variability of the AIDS virus, such immune responses have little, or even no, interest from the point of view of a vaccine.

There exists, therefore, a need for a vaccine capable of inducing neutralizing immunity against primary isolates of HIV.

The applicant has demonstrated, surprisingly, that a chemically modified envelop glycoprotein of HIV makes it possible to attain this objective.

The present invention relates, therefore, to an envelope glycoprotein of HIV, which is in purified form and can be obtained by a method comprising the following steps:

(1) production of an envelope glycoprotein in purified form,

(2) reduction of at least one disulfide bridge of the glycoprotein of step (1),

(3) alkylation of at least two free sulfhydryl groups,

(4) optionally, oxidation of the remaining free sulfhydryl groups,

(5) denaturation and

(6) renaturation.

According to one particular embodiment, the glycoprotein (1) is in dimeric form and corresponds preferably to a gp160MN/LAI. According to one particular embodiment step (2) is carried out by adding a reducing agent according to a (moles of reducing agent)/(moles of sulfhydryl groups) molar ratio of 1 to 500.

According to another particular embodiment step (3) is carried out by adding an alkylating agent according to a (moles of alkylating agent)/(moles of sulfhydryl groups) molar ratio of 1 to 1000. According to one particular embodiment NEM is used as alkylating agent according to a (moles of NEM)/(moles of sulfhydryl groups) molar ratio of 1 to 100, preferably 10.

According to another aspect, the present invention relates to a composition comprising a mixture of chemically modified proteins as defined above.

According to another aspect, the present invention relates to an antibody directed against a chemically modified envelope glycoprotein as defined above, this antibody being preferably monoclonal.

According to a fourth aspect, a subject of the present invention is a vaccine against HIV comprising:

(a) a chemically modified envelope glycoprotein as defined above or a composition as defined above, or an antibody as defined above or a mixture of these antibodies,

(b) a pharmaceutically acceptable support or diluent and

(c) optionally, an adjuvant or mixture of adjuvants.

According to one particular embodiment, the vaccine according to the invention is used for inducing antibodies which neutralize HIV in a human individual, therapeutically or prophylactically.

According to another aspect, the present invention relates to a diagnostic method comprising bringing a biological fluid into contact with an antibody as defined above, and determining the immune complexes thus formed.

The other characteristics and advantages of the present invention will appear in the detailed description which follows.

In the context of the present invention, the term “envelope glycoprotein” is intended to mean a glycosylated gp160, gp120 or gp140 protein. The envelope protein is in monomeric, dimeric or multimeric form; it will be preferably in dimeric form. This envelope protein may or may not be a recombinant protein, and may also consist of a hybrid protein; the term “hybrid” being used herein in its conventionally accepted sense, namely a protein comprising sequences originating from envelope proteins of various strains of laboratory-adapted viruses or of primary isolates of HIV. Envelope proteins in which the amino acid sequence differs from that of the native protein by mutation(s), deletion(s), insertion(s) or substitution(s) of amino acid(s) are also included in the definition above provided that these modifications do not abolish the formation of antibodies which can neutralize primary isolates of HIV. This characteristic can be easily determined using the test provided in the present application. In the context of the present invention, use is made preferably of gp160MN/LAI as described in example 1 below.

The envelope glycoprotein of step (1) is used in substantially purified, isolated form. The expression “isolated and substantially purified protein” is intended to mean a protein having a degree of purity of at least 75%, preferably of at least 80%, as determined by the method of acrylamide gel electrophoresis (SDS PAGE) (LAEMMLI U. K. 1970. Nature 27: 680-685.) and analysis by densitometry. In the present application, such a protein is referred to under the term “protein in purified form”. Diverse methods for purifying the envelope protein, which may be natural or recombinant, of HIV have been described in the literature. Reference may be made, for example, to the articles by Pialoux et al. (Aids Res. Hum. Retr., 11, 373-381, 1995) and by Sakmon-Ceron et al. (Aids Res. Hum. Retr., 12, 1479-1486, 1995) or to the text WO 91/13906.

With regard to the recombinant proteins, it should be noted that the glycoproteins thus purified have interchain disulfide bridges, whatever the nature of the host or of the vector used. The glycoproteins thus associate with each other in part as covalent dimers which are visible on SDS PAGE gel (Owens R J. Compans R W. Virology, 179 (2): 827-833, December 1990).

The envelope glycoprotein in purified form is subjected, firstly, to a step of partial or total reduction of the intrachain and/or interchain disulfide bridges, in which at least one disulfide bridge is reduced.

The reduction step is carried out by reacting the envelope glycoprotein of step (1) with a reducing agent, at room temperature and with gentle stirring. The reducing agent can be chosen from dithiothreitol (DTT), beta-mercaptoethanol, reduced glutathione and sodium borohydride molecules, for example. The amount of reducing agent, expressed as the molar ratio (moles of reducing agent)/(moles of sulfhydryl groups), varies between 1 and 0.5×10⁴ and corresponds preferably to a molar ratio of 50. The reduction is carried out at a basic pH of 7 to 10, preferably at a pH of 7.8. Control of the pH value is obtained by adding a buffer; any buffer which is suitable for this purpose can be used. A sodium phosphate buffer is preferably used. By way of indication, in the case of DTT, the reaction is carried out for approximately 15 minutes, the molar ratio moles of DTT/moles of SH used is from 1 to 0.5×10⁴, and preferably 50.

The duration of the reduction reaction is variable and depends on the molar ratio and reducing agent chosen.

The reduction reaction conditions which allow the reduction of at least one disulfide bridge can be easily determined by those skilled in the art using the teaching provided herein. The reduction can be controlled by SDS PAGE analysis since the reduction of the interchain disulfide bridges transforms the dimers into monomers. Finer controls for this reduction are possible using ¹⁴C-labeled N-ethylmaleimide (NEM), or more simply using a calorimetric assay based on dithio-nitrobenzoic acid (DTNB).

The free sulfhydryl groups thus obtained are then subjected to an alkylation reaction in which the product from step (2) reacts with an alkylating agent.

In the context of the present invention, the term “alkylating agent” is intended to mean any reagent capable of reacting specifically with —SH groups so as to give a covalent bond. By way of illustration, mention may be made of: N-ethylmaleimide, iodo-acetamide. The amount of alkylating agent used, expressed as the molar ratio (moles of alkylating agent)/(moles of sulfhydryl groups), is from 1 to 100, preferably from 10 to 100. It is necessary to take care to have an excess of alkylating agent with respect to the reducing agent so as to neutralize the action of the latter.

The alkylation reaction is carried out at a pH of 6 to 8, preferably at a pH of 7, at room temperature. Control of the pH value is obtained by adding a buffer; any buffer suitable for this purpose can be used. A sodium phosphate buffer is preferably used.

The alkylation reaction conditions which allow the alkylation of at least two —SH groups can be easily determined by those skilled in the art using the teaching provided herein. The alkylation can be controlled using ¹⁴C-NEM as is described below in the examples.

The product derived from step (3) can be subjected to an oxidation step during which the remaining free sulfhydryl groups are oxidized in the presence of an oxidizing agent. If free sulfhydryl groups are still present at the end of step (3), an oxidation step is preferably carried out before the denaturation step.

In the context of the present invention, the term “oxidizing agent” is intended to mean any molecule linked by disulfide bridges, such as oxidized glutathione or cystine, but it may also be other molecules such as quinones, oxygen, etc. By way of illustration, mention may be made of the mixture reduced glutathione/oxidized glutathione. In this mixture, the reduced glutathione allows the disulfide bridges to dissociate in order to reassociate in a more stable thermodynamic state.

The oxidation reaction is carried out at a pH of 7 to 9, preferably at pH 7.8, at a temperature of 4 to 25° C. The oxidizing agent is used according to a (moles of oxidizing agent)/(moles of sulfhydryl groups) molar ratio of 50 to 5 000, preferably of 500. By way of illustration, when the mixture reduced glutathione/oxidized glutathione is used, the reaction is carried out with an oxidized glutathione content from 1 to 1 000 times higher than the reduced glutathione content. For example, a ratio of 500 oxidized glutathione molecules per mole of gp160MN/LAI can be advantageously used.

The duration of the oxidation step can vary between 5 minutes and 24 hours, and corresponds preferably to 30 minutes. The oxidation reaction conditions which allow the oxidation of the free sulfhydryl groups can be easily determined by those skilled in the art using the teaching provided herein. The oxidation can be controlled by a method similar to that used for controlling the reduction step, taking great care with the positive controls of the test.

The product derived from step (3) or (4) is then denatured by the action of one or more denaturing agent(s) used in a proportion of 0.1 to 5% (weight/vol) so as to modify the conformation of the glycoprotein. For this purpose, one or more detergent(s), preferably ionic detergent(s), or one or more chaotropic agent(s), can be used, for example. By way of illustration, mention may be made of the following ionic detergents: the salts of dodecyl sulfate, in particular sodium dodecyl sulfate (SDS) or lithium dodecyl sulfate, the salts of dioctyl sulfosuccinate (sodium dioctyl sulfosuccinate, for example), the salts of cetyltrimethylammonium (bromine cetyltrimethylammonium, for example) DTAB, the salts of cetylpyridinium (chlorine cetylpyridinium, for example), the N-dodecyl-or N-tetradecylsulfobetaines, the zwittergents 3-14, and 3-[(3-cholamidopropyl)dimethylamino]-1-propane sulfonate (CHAPS), and the following neutral detergent(s): tween20®, tween80®, octylglucoside, laurylmaltoside, hecameg®, lauryldimethylamine, decanoyl-N-methylglucamide, polyethylene glycol lauryl ether, triton X100®, Lubrol PX®, for example. By way of example, urea, guanidine and sodium thiocyanate may be mentioned as chaotropic agents which can be used in the context of the present invention.

In the context of the present invention, SDS is preferably used, in particular at a concentration of 0.1% (weight/vol.).

The denaturation reaction is carried out at neutral or alkaline pH, at room temperature.

The denaturation reaction conditions which allow conformational modifications of the molecule can be easily determined by those skilled in the art using the teaching provided herein. The denaturation can be controlled by spectrophotometric measurement, measuring the absorbence of the tyrosine, phenylalanine and tryptophan residues of the molecule, or by circular dichroism.

The glycoprotein thus denatured is then subjected to a renaturation step which can be implemented by dialysis against 1 000 volumes of a detergent-free buffer, preferably a phosphate buffer containing sodium chloride (PBS). The effectiveness of the dialysis step can be easily determined by calorimetric analysis of the residual oxidizing agents or by HPLC, by showing the disappearance of certain reagents used for manufacturing the antigen. By way of illustration, the dialysis can be carried out overnight at room temperature, with gentle stirring, against a PBS buffer.

According to one preferred embodiment, the gp160MN/LAI in purified form (1) is chemically modified by a method comprising the steps of: (2) reduction by incubation with DTT according to a (moles of DTT)/(moles of SH groups) molar ratio of 50, at a pH of 7, for a duration of approximately 15 minutes at room temperature, (3) alkylation by incubation with NEM according to a (moles of NEM)/(moles of SH groups) molar ratio of 10, at a pH of 7, for a duration of approximately 15 minutes at room temperature, (4) oxidation by incubation of the product of step (3) with a reduced glutathione/oxidized glutathione mixture according to a (moles of oxidized glutathione)/(moles of SH groups) molar ratio of 500, with a reduced glutathione/oxidized glutathione ratio of 10, at a pH of 7.8, for a duration of approximately 30 minutes, (5) denaturation of the product of step 4 by incubation with 0.1% of SDS (weight/vol.) for a duration of approximately 15 minutes and at a pH of 7.8, then (6) renaturation by dialysis against a PBS buffer overnight at room temperature.

According to another aspect, the present invention relates to a composition comprising a mixture of chemically modified glycoproteins as defined above. In such a case, these chemically modified glycoproteins can differ, for example, by the nature of the constituent envelope glycoprotein (for example the glycoproteins originating from various strains or primary isolates, some possibly also corresponding to hybrid proteins) or by their method of preparation, the parameters of the latter, such as the concentration and the nature of the reagents, possibly varying. Any conceivable mixture comprising one or more chemically modified envelope glycoprotein(s) is included in the scope of the present invention.

A subject of the present invention is also the antibodies directed against the chemically modified envelope glycoproteins as described above. The preparation of such antibodies is carried out by the conventional techniques for producing polyclonal or monoclonal antibodies (Kohler G et al. European Journal of Immunology. 6(7): 511-9, July 1976).

These antibodies are particularly suitable for being used in a passive immunization scheme.

A subject of the present invention is also vaccines which are useful for therapeutic and prophylactic purposes. The vaccines according to the present invention comprise a chemically modified envelope glycoprotein as defined above or a mixture of such glycoproteins, a pharmaceutically acceptable support or diluent and, optionally, an adjuvant.

The vaccine according to the present invention can, therefore, contain a single type of chemically modified envelope glycoprotein or a mixture of diverse types of chemically modified envelope glycoprotein as defined above.

According to another aspect, the vaccine according to the present invention comprises anti-chemically modified envelope glycoprotein antibodies. In this case also, any mixture of antibodies, monoclonal or polyclonal, directed against various parts of the same chemically modified envelope glycoprotein or against various chemically modified envelope glycoproteins forms part of the present invention.

The amount of chemically modified envelope glycoprotein in the vaccine according to the present invention depends on many parameters, as will be understood by those skilled in the art, such as the nature of the chemically modified glycoprotein, the route of administration and the condition of the person to be treated (weight, age, clinical condition, etc.). A suitable amount is an amount such that a humoral immune response capable of neutralizing primary isolates of HIV is induced after administration of the latter. The vaccines according to the present invention can also contain an adjuvant. Any pharmaceutically acceptable adjuvant or mixture of adjuvants can be used for this purpose. By way of example, mention may be made of the salts of aluminum, such as aluminum hydroxide or aluminum phosphate. Conventional auxiliary agents, such as wetting agents, fillers, emulsifiers, buffers, etc. can also be added to the vaccine according to the invention.

The vaccines according to the present invention can be prepared by any conventional method known to those skilled in the art. Conventionally, the antigens are mixed with a pharmaceutically acceptable support or diluent, such as water or phosphate buffered saline solution. The support or diluent will be selected as a function of the pharmaceutical form chosen, of the method and route of administration, and of the pharmaceutical practice. The suitable supports or diluents and the requirements regarding pharmaceutical formulation are described in detail in Remington's Pharmaceutical Sciences, which represents a work of reference in this field.

The vaccines mentioned above can be administered via any conventional route, usually employed in the field of vaccines, such as the parenteral (intravenous, intramuscular, subcutaneous, etc.) route. The administration can be carried out by injecting a single dose or repeated doses, for example on D0, at 1 month, at 3 months, at 6 months and at 12 months. Injections on D0, at 1 month and at 3 months will be preferably used.

The present invention is also intended to cover a chemically modified envelope glycoprotein as defined above and the vaccine containing such a glycoprotein or a mixture of such glycoproteins, for their use in order to induce antibodies which can neutralize primary isolates of HIV.

The applicant has demonstrated, surprisingly, that the chemically modified envelope glycoproteins according to the invention are capable, after administration, of inducing antibodies which can neutralize primary isolates of HIV. These antigens represent, therefore, valuable candidates for the development of a vaccine which can be used for protecting and/or treating a large number, or even all, of the individuals at risk or infected with HIV.

The present invention will be described in more detail in the following examples.

The examples described below are given purely by way of illustration of the invention and can in no way be considered as limiting the scope of the latter. For the purposes of clarity, the examples are limited to chemically modified envelope glycoproteins consisting of gp160MN/LAI.

### EXAMPLE 1

### Preparation of the Glycoprotein gp160MN/LAI

The glycoprotein gp160MN/LAI is a soluble hybrid glycoprotein in which the gp120 subunit derives from HIV-1MN and the gp41 subunit derives from the LAI isolate. The DNA sequences corresponding to these two components are fused with the aid of an SmaI restriction site which modifies neither the amino acid sequence of gp120 nor that of gp41. The preparation of this protein is described below.

The sequence encoding gp120MN is amplified by PCR from SupT1 cells infected with HIV MN, using oligonucleotides which introduce the SphI and SmaI restriction sites, respectively, immediately downstream of the sequence encoding the leader peptide and upstream of the cleavage sites located between gp120 and gp41. The sequence encoding the gp41 subunit is produced in the following way: the complete sequence encoding the env protein of HIV-1 LAI is placed under the control of the pH5R promoter of the vaccinia virus. Several modifications are introduced into this coding region. An SphI restriction site is created immediately downstream of the sequence encoding the leader peptide, without modifying the amino acid sequence. An SmaI restriction site is created immediately upstream of the sequence encoding the cleavage sites located between gp120 and gp41, without modifying the amino acid sequence. The two cleavage sites at position 507-516 (amino acids numbered according to the method of Myers et al., described in Human retroviruses and AIDS (1994) Los Alamos National Lab. (USA)) were mutated (i.e. the sequence of origin KRR . . . REKR was mutated to QNH . . . QEHN). The sequence encoding the hydrophobic transmembrane peptide IFIMIVGGLVGLRIVFAVLSIV (i.e. amino acids 689-710 according to Myers et al., above) was deleted. Finally, the second E codon of the sequence encoding PEGIEE (i.e. amino acids 735-740 according to Myers et al. (above)) was replaced with a stop codon, corresponding to the 29th amino acid of the intracytoplasmic domain.

The plasmid into which the LAI sequence is inserted between the homologous regions of the vaccinia virus TK gene is cleaved with SphI and SmaI, and then ligated to the sequence of the gp120MN. The virus VVTG9150 is then constructed by conventional homologous recombination.

The recombinant vector of the vaccinia virus, VVTG9150, thus produced is used for producing the gp160MN/LAI. For this purpose, the vector is propagated on BHK21 cells. The gp160MN/LAI-2 is produced on BHK21 cells infected for 72 hours with the recombinant vaccinia virus VVTG9150. After culturing in a biogenerator, the supernatant is harvested, filtered and ultrafiltered to give the concentrated harvest. The purification takes place in three steps. Some contaminants of the gp160MN/LAI-2 are attached to an anion exchange column. The nonattached fraction is chromatographed on an immunoaffinity column using a monoclonal antibody. After elution, the gp160MN/LAI-2 is desalted by gel filtration chromatography in PBS buffer. In order to inactivate the residual vaccinia, the glycoprotein is heated at 60° C. for 1 hour before being filtered to give the purified antigen.

The concentration of the gp160MN/LAI-2 used for preparing the chemically modified proteins is 1 mg/ml of proteins (determined by calorimetric assay, BCA kit, Pierce™), and it is 77% pure (determined by SDA PAGE electrophoresis and optical densitometry analysis using the ScannerGS700 from Biorad™). The glycoprotein is in a phosphate buffer with the following composition: 137 mM NaCl; 2.7 mM KCl; 6.5 mM Na₂HPO₄; 1.5 mM KH₂PO₄; pH 7.4 (PBS).

The gp160MN/LAI-2 thus obtained has a molecular weight of 140 kD by SDS-PAGE.

### EXAMPLE 2

### Preparation of Chemically Modified Glycoproteins According to the Invention

Starting with 172 μl of purified gp160 (1 mg/ml), 21 μl of 1M sodium phosphate buffer, pH 7.8; 2 μl of distilled water and 19.5 μl of 50 mM dithiothreitol (DTT) are added, and the mixture is vortexed for 15 s and incubated for 15 min at 25° C. 16 μl of 1M sodium phosphate buffer (NaH₂PO₄) are added in order to lower the pH to 7, the sulfhydryl groups are blocked by adding 14 μl of 100 mM N-ethylmaleimide (NEM), and the mixture is incubated for 15 min at 25° C. The sulfhydryl groups are re-oxidized by adding sodium phosphate buffer at pH 7.8, a mixture of 4.8 μl of 150 mM reduced glutathione and 71.6 μl of 100 mM oxidized glutathione is added, and the mixture is incubated for 30 min at 25° C. The gp160 dimers are then dissociated by adding 12 μl of 3% sodium dodecyl sulfate (SDS). The sample is placed in a dialysis cassette with a capacity of 3 ml, against 1 000 volumes of PBS buffer (without detergent). The dialysis is performed overnight at room temperature with gentle stirring. The gp160 molecules thus treated are in the form of monomers and dimers. The protein thus obtained is named BA29.

A glycoprotein BA29(7.8) is prepared according to the procedure as described above, in which the pH of 7 of the NEM alkylation step is replaced with a pH of 7.8.

The SDS PAGE analysis under reducing conditions (DTT), obtained with the gp160 which is dialyzed and, where appropriate, fixed with the bifunctional bridging agent ethylene glycol bis(succinimidyl succinate) (EGS), shows the presence of monomers and of dimers in the dialyzed gp160 solution.

### EXAMPLE 3

### Preparation of Chemically Modified Glycoproteins with Variation of the Concentration of Alkylating Agent

In order to determine the role of the alkylating agent which is used for preparing the proteins according to the invention, several chemically modified glycoproteins were prepared in the presence of various concentrations of alkylating agent.

The preparation BA53 is produced according to the method described in example 2 for BA29, in which the NEM has been omitted.

The preparation BA55 is produced according to the method described in example 2 for BA29, in which the concentration of NEM used is 10 times lower than that indicated in example 2. The preparation BA56 is produced according to the method described in example 2 for BA29, in which the concentration of NEM used is 10 times higher than that indicated in example 2.

These antigens were prepared in parallel, to be injected into animals and for a biochemical measurement of the amount of NEM attached per molecule of gp160. For this, ¹⁴C-labeled NEM was used. Approximately 4 MBq of ¹⁴C NEM were added per mM of nonradioactive NEM. The radioactivity measured is then directly proportional to the concentration of NEM. During the final dialysis step, it was verified that the radioactive NEM had been thoroughly eliminated and that only the radioactivity covalently attached to the protein remained in the sample.

Aliquots of the antigens manufactured according to the various protocols were then placed in scintillation vials and the β-radiation emitted by the disintegration of the ¹⁴C atoms was recorded for one minute. The counts of radioactivity are directly proportional to the amount of NEM attached. Since the amount of gp160 present in each aliquot was known, the ratio of the number of NEM molecules per gp160 molecule could be established.

The results obtained show that NEM cannot become attached to the nonreduced gp160 (control). 8 molecules of NEM per molecule of gp160 become attached to the gp160 treated according to the invention (BA29). Consequently, there are at least 4 modified disulfide bridges inside this antigen. It was shown that the use of a ten-fold lower concentration of NEM (BA55) made it possible to attach the NEM to only 2 (1.6 to 1.8) sulfhydryls per gp160 molecule. It is possible that a sole disulfide bridge is modified inside this antigen. It was shown that the use of a ten-fold higher concentration of NEM (BA56) abolished the immunological properties of the molecule.

### EXAMPLE 4

### Analysis of the Immunogenicity of the Chemically Modified Glycoproteins in Guinea Pigs

Formulation of the Chemically Modified Glycoproteins

The chemically modified glycoproteins are diluted sterilely in stabilizing medium, and then adsorbed on aluminum phosphate. The stabilizing mixture is composed of a mixture of amino acids and of Dulbecco's Modified Eagle Medium DMEM-F12 (Gibco, France). The chemically modified glycoproteins prepared in examples 2-4 (BA29, BA29(7.8), BA52, BA53, BA55 and BA56) are diluted in the stabilizing mixture, before adding an equal volume of aluminum phosphate at 6.3 mg/ml in PBS to this mixture.

The chemically modified glycoproteins named BA53 and BA52 are obtained using the method described in example 2 for BA29, in which the NEM alkylation step has been eliminated (preparation BA53), or the SDS denaturation step has been eliminated (preparation BA52).

Immunizations

For each chemically modified glycoprotein, a group of 5 Dunkin-Hartley albino female guinea pigs (Charles River) weighing 400 g are used. Each guinea pig receives 5 μg of antigen via the intramuscular route, in the right and left thighs (0.5 ml in each thigh) on D1 and on D29. A 3 ml volume of blood is taken by cardiac puncture under anesthesia on days −1, 28 and 56 (final bleed approximately 30 ml).

Titrations of the Sera, by ELISA, Against gp160MN/LAI

The guinea pig sera thus obtained were analyzed by ELISA assay against the native gp160MN/LAI. The gp160MN/LAI is immobilized on the solid phase in a proportion of 130 ng per cupule for 1 hour at 37° C. The plate is emptied and then saturated with a PBS, 0.1% Tween 20 buffer containing 5% of powdered skimmed milk. Each serum is diluted on the plate according to 3-fold serial dilutions, between 1/100^{th} and 1/100 000^{th} depending on the case, in saturation buffer, and incubated for 1 hour 30 at 37° C.

A peroxidase-coupled rabbit anti-guinea pig antibody (Sigma, St Louis), diluted 3 000-fold, makes it possible to reveal the presence of antibodies specific for the gp160MN/LAI. The titers are calculated automatically by the reader from the optical density reed and from the straight line obtained with a calibration serum. The mean values of the titer of the immunoglobulins specific for the gp160MN/LAI for each group of guinea pigs are between 10⁵ and 10⁶.

The control group injected with the nontreated gp160MN/LAI is identified under the code BA1. The preparations BA55 and BA29 induce specific antibodies, BA29 giving a titer greater than 5×10⁵. No antibody specific for the gp160MN/LAI was detected in the preimmune sera.

These results show clearly that the chemically modified glycoproteins according to the present invention are capable of inducing antibodies which recognize specifically the envelope glycoprotein of HIV.

### EXAMPLE 5

### Test for Neutralization of Primary Isolates of HIV

The tests for neutralization of primary isolates of HIV were carried out on the isolates Bx17 and T051 using the method of C. Moog et al., as described in AIDS Res. Hum. Retroviruses, 1997, 13, 19-27, all of which is incorporated herein by way of reference.

This test was carried out against several primary isolates of HIV-1; only the results obtained with the isolates Bx17 and T051 are detailed herein.

The results obtained show clearly that the chemically modified glycoproteins according to the resent invention are superior to the glycoproteins which were nontreated or subjected to different treatments, and allow the neutralization of primary isolates of the virus, even though they are obtained from a gp160 isolated from a laboratory-adapted strain (TCLA). Under these conditions, it may reasonably be thought that the mixtures of chemically modified glycoproteins according to the invention may cause the neutralization of many primary isolates of HIV.

The results obtained are summarized in table 1 below:

Table 1: Titer for Neutralization of Primary Isolates of HIV1 Viruses, Expressed as the Inverse of the Dilution

| Serum | B × 17 | T051 |
|---|---|---|
| BA1 | NN | NN |
| BA29 | 7 | 10 |
| BA52 | NN | NN |
| BA53 | NN | Not determined |
| BA55 | 10 | Not determined |
| BA56 | NN | Not determined |

NN: Non neutralizing

The numbers indicate the inverse of the dilution of the serum for which neutralization was observed.

### CONCLUSIONS

The antigens according to the present invention are manufactured using a gp160MN/LAI of a laboratory-adapted HIV-1 virus. However, these antigens make it possible to induce, in the animal immunized, a humoral response capable of neutralizing primary isolates of the HIV-1 virus, which constitutes progress with respect to the current knowledge on this subject.

1. An envelope glycoprotein of HIV, which is in purified form and can be obtained by a method comprising the following steps:
(1) production of an envelope glycoprotein in purified form,
(2) reduction of at least one disulfide bridge of the glycoprotein of step (1),
(3) alkylation of at least two free sulfhydryl groups,
(4) optionally, oxidation of the remaining free sulfhydryl groups,
(5) denaturation and
(6) renaturation.

2. The glycoprotein as claimed in claim 1, in which the glycoprotein (1) is in dimeric form.

3. The glycoprotein as claimed in claim 2, in which the glycoprotein of step (1) is a gp160MN/LAI.

4. The glycoprotein as claimed in any one of claims 1 to 3, in which step (2) is carried out by adding a reducing agent according to a (moles of reducing agent)/(moles of sulfhydryl groups) molar ratio of 1 to 500.

5. The glycoprotein as claimed in claim 4, in which DTT is used as a reducing agent according to a (moles of reducing agent)/(moles of sulfhydryl groups) molar ratio of 50.

6. The glycoprotein as claimed in any one of claims 1 to 3, in which step (3) is carried out by adding an alkylating agent according to a (moles of alkylating agent)/(moles of sulfhydryl groups) molar ratio of 1 to 1 000.

7. The glycoprotein as claimed in claim 6, in which NEM is used as an alkylating agent according to a (moles of NEM)/(moles of sulfhydryl groups) molar ratio of 1 to 100, preferably 10.

8. The glycoprotein as claimed in any one of claims 1 to 7, in which the denaturation step is carried out by adding an ionic detergent in an amount of 0.1 to 5% (weight/vol), preferably SDS in an amount of 0.1% (weight/vol).

9. The glycoprotein as claimed in any one of claims 1 to 8, in which the gp160MN/LAI in purified form (1) is chemically modified by: (2) reduction by incubation with DTT according to a (moles of DTT)/(moles of SH groups) molar ratio of 50, at a pH of 7, for a duration of approximately 15 minutes at room temperature, (3) alkylation by incubation with NEM according to a (moles of NEM)/(moles of SH groups) molar ratio of 10, at a pH of 7, for a duration of approximately 15 minutes at room temperature, (4) oxidation by incubation of the product of step (3) with a reduced glutathione/oxidized glutathione mixture according to a (moles of oxidized glutathione)/(moles of SH groups) molar ratio of 500, with a reduced glutathione/oxidized glutathione ratio of 10, at a pH of 7.8, for a duration of approximately 30 minutes, (5) denaturation of the product of step 4 by incubation with 0.1% of SDS (weight/vol.) for a duration of approximately 15 minutes and at a pH of 7.8, then (6) renaturation by dialysis against a PBS buffer overnight at room temperature.

10. A composition comprising a mixture of glyco-proteins as claimed in any one of claims 1 to 9.

11. An antibody directed against the glycoprotein as claimed in any one of claims 1 to 9.

12. A vaccine against HIV comprising:
(a) a chemically modified envelope glycoprotein as claimed in any one of claims 1 to 9 or a composition as claimed in claim 10, or an antibody as claimed in claim 11 or a mixture of these antibodies,
(b) a pharmaceutically acceptable support or diluent and
(c) optionally, an adjuvant or mixture of adjuvants.

13. The vaccine as claimed in claim 12, comprising a chemically modified envelope glycoprotein as claimed in any one of claims 1 to 9 or a composition as claimed in claim 10, for its use in order to induce antibodies which neutralize HIV in a human individual, therapeutically or prophylactically.
